(19) United States
(12) Patent Application Publication
(10) Pub. No.: US 2023/0036051 A1
(43) Pub. Date: Feb. 2, 2023

(54) CANNABINOID STORAGE STABILITY

(71) Applicant: Resurgent Biosciences, Inc., Minneapolis, MN (US)

(72) Inventors: Justin Bueno, Minneapolis, MN (US); Eric Greenbaum, New York, NY (US)

(73) Assignee: Resurgent Biosciences, Inc., Minneapolis, MN (US)

(21) Appl. No.: 17/381,613

(22) Filed: Jul. 21, 2021

(51) Int. Cl. A61K 9/14; A61K 31/05; A61K 31/352; A61K 47/10; A61K 47/18; A61J 1/14

(52) U.S. Cl. CPC A61K 9/145; A61K 31/05; A61K 31/352; A61K 47/10; A61K 47/183; A61J 1/1412; A61K 9/4816

(57) ABSTRACT

Materials and methods for improving the shelf-life of cannabinoids are described. For example, methods of improving the stability of cannabinoids during storage by applying one or more exogenous terpenes, terpenoids, or blend thereof to a cannabinoid-containing product, and packaging configured to stabilize a cannabinoid-containing product by preserving the applied one or more terpenes, terpenoids, or blend thereof are provided.

### BACKGROUND

Cannabis for both recreational and medicinal uses have increased in popularity in the United States and around the world. While the major cannabinoids (THC and CBD) receive most of the attention, terpenes, terpenoids, flavonoids, and other volatile components that impart flavor and aroma to cannabis and are also recognized to have important pharmacological effects.

The stability of different cannabinoids and volatile components in cannabis-based products depends on the storage form and storage conditions. Exposure to high or low humidity, extreme temperatures, light, or air can reduce product quality, potency, cannabinoid concentration and flavor over time. For example, air oxidation is known to lead to significant losses in THC content in plant material, extracts and isolates, decreasing the value or perceived value of the product. Yet, bulk-storage containers must be opened to access product. After 3 months storage at 25° C. in a closed container, at least 2% (mass fraction) of the total THC content (THC+0.877*THCA) is expected to be lost. Moreover, after 1 year of storage at 30° C. in a closed container, at least 22% of the total THC content is expected to be lost, indicating that the duration and temperature of storage mediates the degradation. The instability of cannabinoids creates a need for ongoing product monitoring: from raw feedstock, to intermediate points in the preparation of applied consumables (solvents, filters, etc.), to final product. Post-market surveillance is not practical to monitor product quality and potency in the days, weeks and months after production. Thus, there is a need for improved methods and packaging to stabilize the cannabinoid profile and ensure product quality and potency during storage.

### SUMMARY OF THE INVENTION

The present disclosure describes materials and methods for improving the shelf-life of cannabinoids. In particular, methods of improving the stability of cannabinoids during storage using one or more terpenes, terpenoids, or a blend thereof, and/or packaging configured to stabilize a cannabinoid-containing product by preserving the one or more terpenes, terpenoids, or blend thereof are provided.

A first aspect of the present disclosure features a method of improving stability of at least one cannabinoid of a cannabinoid-containing product during storage comprising: (a) identifying a cannabinoid-containing product that will be stored for a predetermined duration; (b) contacting the cannabinoid-containing product with one or more exogenous terpenes, terpenoids, or a blend thereof; and (c) storing the cannabinoid-containing product for the predetermined duration; whereby a final content of at least one cannabinoid after storing is substantially similar to an initial content of the at least one cannabinoid. The exogenous terpene or terpenoid can be selected from the group consisting of 7,8-dihydroionone, Acetanisole, Acetyl Cedrene, Anethole, Anisole, Benzaldehyde, Bergamotene (α-cis-Bergamotene) (α-trans-Bergamotene), α-Bisabolol, Bornyl acetate, Borneol, Butanoic, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene)β-Caryophyllene), Caryophyllene oxide, Castoreum Absolute, Cedrene (α-Cedrene) β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), D-Limonene, Decanal, Dehydrovomifoliol, Diallyl Disulfide, Dihydroactinidiolide, Dimethyl Disulfide, Eicosane/Icosane, Elemene (β-Elemene), Estragole, Ethyl acetate, Ethyl Cinnamate, Ethyl maltol, Eucalyptol, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10), 11-diene, Guaiol, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (β-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, α-Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpineol, α-Terpinene, γ-Terpinene, Terpinolene, Thiophenol, Thujone, Thymol, α-Tocopherol, Tonka Undecanone, Undecanal, Valencene, Valeraldehyde/Pentanal, Verdoxan, α-Ylangene, Umbelliferone, Valencene and Vanillin, or a blend thereof comprising at least two terpenes, at least two terpenoids, or at least one terpene and at least one terpenoid. The cannabinoid-containing product can be contacted with a blend including a mixture of at least four exogenous terpenes, terpenoids, or a combination thereof. The method can include adding 0.01-5% one or more exogenous terpenes, terpenoids, or a blend thereof by weight of the cannabinoid-containing product. The cannabinoid-containing product can be contacted with a mixture of volatile terpenes, volatile terpenoids, or a mixture thereof optionally comprising Eucalyptol, α-Terpinene, Limonene, and p-Cymene, or a mixture of non-volatile terpenes, non-volatile terpenoids, or a mixture thereof optionally comprising δ-Terpinene, Geraniol, Carvone, Linalool, and Thymol. The duration of storage can be at least 2 months. The at least one cannabinoid can be selected from the group consisting of tetrahydrocannabiniol in acid or decarboxylated form, cannabidiol in acid or decarboxylated form, cannabigerol in acid or decarboxylated form, cannabichromene in acid or decarboxylated form, tetrahydrocannabivarin in acid or decarboxylated form, Cannabidivarin in acid or decarboxylated form, and cannabinol in acid or decarboxylated form. The contacting step can include exposing the cannabinoid-containing product to a headspace comprising the terpene, terpenoid, or blend thereof for the duration of storage. Contacting can occur during preparation, manufacture, or packaging of the product.

A second aspect of the present disclosure features a dispersion matrix for use in packaging a cannabinoid-containing product, comprising a terpene, terpenoid, or a blend thereof configured to improve stability of at least one cannabinoid of the cannabinoid-containing product during storage. The dispersion matrix can further include an adhesive component configured to affix the matrix to an interior surface of a package adapted to hold the cannabinoid-containing product. The dispersion matrix can further include a core containing the terpene, terpenoid, or blend thereof. The core can be formulated as an oil based gel or impregnated with solution comprising terpene, terpenoid, or blend thereof. The core can be seated within a capsule body. The dispersion matrix can further include at least one material configured to controllably release the terpene, terpenoid, or blend thereof, over time. The material can be a laminate, silicone rubber, or a control release polymer. The material can be a multi-laminate sustained release device capable of releasing vapor at a controlled rate for a prolonged period of time.

A third aspect of the present disclosure features a system for storing a cannabinoid-containing product, the system comprising: (a) a storage container configured to hold a cannabinoid-containing product; (b) a dispersion matrix comprising an exogenous terpene, terpenoid, or blend thereof for improving the stability of at least one cannabinoid; and (c) a lid to seal the storage container; wherein the dispersion matrix and the cannabinoid-containing product are in communication with the air in the package allowing the exogenous terpene, terpenoid, or blend thereof to be in contact with the cannabinoid-containing product. The system can further include a humidity controlling element. The lid can be configured to secure the dispersion matrix in the storage container.

These and other systems, methods, objects, features, and advantages of the present disclosure will be apparent to those skilled in the art from the following detailed description of the embodiments and drawings.

### BRIEF DESCRIPTION OF THE DRAWINGS

FIGS. 1A-B depict chemical characterizations of oxidative, thermal, and/or light exposure degradation products of cannabinoids: (A) shows Δ9-Tetrahydrocannabinolic acid (Δ9-THCA) is degraded to Cannabinolic acid (CBNA), and Δ9-tetrahydrocannabinol (Δ9-THC) is degraded to Cannabinol (CBN), and (B) shows oxidized derivatives of Δ9-THC, which had been stored in an ethanol/propylene glycol solution for 5 years.

FIG. 2 are bar graphs showing the total THC content (% THC+(% THCA*0.877) over time under Non-Accelerated Storage Conditions without exogenous terpenes (“Control”), under Accelerated Conditions (with the oxidative agent hydrogen peroxide), without exogenous terpenes (“H₂O₂”), and under Accelerated Conditions in the presence of exogenous terpenes (“H₂O₂+Terpene”). Experiments were performed in triplicate; All data are expressed as the mean. ±standard deviation.

FIG. 3 shows the concentration-dependence of the relative increase in THC content (%) over control after 81 days storage in the presence of a volatile Terpene Blend. Experiments were performed in triplicate; Data are expressed as the mean. ±standard deviation.

FIG. 4 shows the concentration-dependence of the relative increase in THC content (%) over control after 81 days storage in the presence of a non-volatile Terpene Blend. Experiments were performed in triplicate; Data are expressed as the mean. ±standard deviation.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context.

The term “cannabinoid-containing product” means any product that contains a naturally occurring or synthetic cannabinoid, including, but not limited to plant material (any part of a cannabis plant, e.g., leaf, flower and/or bud), and resins, extracts, tinctures, and isolates therefrom, pharmaceutical dosage forms, nutritional supplements, and personal care compositions. The cannabinoid-containing product can be an aerosol (e.g., foam, metered, powder, spray), a chewable form (gummy), a bead, a capsule (e.g., coated, containing coated pellets, extended release, delayed release, containing delayed release pellets, and liquid-filled capsules), impregnated cloth, concentrate, cream, dragee, douche, dressing, elixir, emulsion, enema, extract, fiber (e.g., extended release fiber), film (e.g., extended release, soluble, and orally dissolving strips), solution, suspension, gel, granule (e.g., delayed release, effervescent, reconstitutable, for suspension, and coated granules), chewing gum, implant, inhalant, foam, liposomal formulation, injectable formulations, insert, intrauterine device, jelly, liniment, lipstick, liquid, lotion, lotion/shampoo, lozenge, mouthwash, oil, ointment, paste, pastille, patch, pellet, pill, plaster, poultice, powder (for suspension or metered powder), rinse, salve, shampoo, soap, solution, sponge, spray, suppository, suspension, syrup, tablet (e.g., chewable, extended release, coated, containing particles, delayed release, containing delayed release particles, effervescent, for solution, for suspension, multilayer, and orally disintegrating tablets), tampon, tape, tincture, troche, or wafer. The cannabinoid-containing product can be a product that can be consumed as a food or a drink (AKA, “edibles”), made by addition of cannabis plant material or resin, extract, tincture, or isolate therefrom, into foodstuff (e.g., candy, a candy bar, bread, a brownie, cake, infused butter/oil, cheese, chocolate, cocoa, a cookie, gummy candy, a lollipop, a mint, a pastry, peanut butter, popcorn, a protein bar, rice cakes, yogurt, alcoholic beverages, beer, juice, flavored milk, flavored water, liquor, milk, punch, a shake, soda, tea, and water).

As used in the present disclosure, “terpene” refers to constituents of essential oils comprising an isoprene, CH₂═C(CH₃)—CH═CH₂ building block. Terpene hydrocarbons are classified according to the number of isoprene units. For example, monoterpenes has 2 isoprene units, sesquiterpenes have 3, diterpenes have 4, triterpenes have 6, and tetraterpenes have 8 isoprene units. Terpenes can be further classified as volatile or non-volatile based upon vapor pressure. For example, a “volatile terpene” as described herein has a vapor pressure greater than 1 mm Hg at 25° C.; and a “non-volatile terpene” as described herein has a vapor pressure less than 1 mm Hg at 25° C.

The term “terpenoid”, for purposes of the present disclosure, is intended to cover terpenes having at least one C₅H₈ hydrocarbon unit with one or more points of unsaturation within the chemical structure, and derivatives thereof. Terpenoids can be further classified as volatile or non-volatile based upon vapor pressure. For example, a “volatile terpenoid” as described herein can have a vapor pressure greater than 1 mm Hg at 25° C.; and a “non-volatile terpenoid” as described herein can have a vapor pressure less than 1 mm Hg at 25° C.

The terms “substantially similar” and “substantially the same” as used herein refer to the difference between two values (e.g., weight fraction, % cannabinoid content). Two numerical values that are substantially similar are considered to have little or no statistical significance. Two numerical values that are substantially the same can be defined by a sufficiently high degree of similarity between the values. The difference between the two values can be less than about 20%, less than about 15%, less than about 10%, less than about 5%, less than about 3%, less than about 1%, less than about 0.5% or less than about 0.1% as a function of the value of the reference (e.g., final cannabinoid content/initial cannabinoid content).

### Cannabinoid-Stabilizing Compositions

One aspect of the present disclosure includes compositions for improving the stability of at least one cannabinoid of a cannabinoid-containing product during storage, the composition comprising one or more exogenous terpenes, terpenoids, or a blend thereof. While one or more terpenes or terpenoids can be naturally present in cannabinoid-containing products (i.e., endogenous terpenes/terpenoids), as used herein the term “exogenous” refers to any externally-sourced terpene and/or terpenoid that for use with the product in addition to any naturally-occurring terpenes and/or terpenoids. The exogenous terpene, terpenoid, or blend thereof can be synthetic, or extracted or isolated from plant material (e.g., by using chromatography, distillation, extractions, or similar technique for separating volatile or non-volatile fractions). In some cases, the exogenous terpene or terpenoid “purified”, i.e., in a composition of greater than 70% purity. One or more exogenous terpenes or terpenoids can be selected from 7,8-dihydroionone, Acetanisole, Acetyl Cedrene, Anethole, Anisole, Benzaldehyde, Bergamotene (α-cis-Bergamotene) (α-trans-Bergamotene), α-Bisabolol, Bornyl acetate, Borneol, Butanoic, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), D-Limonene, Decanal, Dehydrovomifoliol, Diallyl Disulfide, Dihydroactinidiolide, Dimethyl Disulfide, Eicosane/Icosane, Elemene (β-Elemene), Estragole, Ethyl acetate, Ethyl Cinnamate, Ethyl maltol, Eucalyptol, Eudesmol (α-Eudesmol) β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10), 11-diene, Guaiol, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (β-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Ally Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, α-Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpineol, α-Terpinene, γ-Terpinene, Terpinolene, Thiophenol, Thujone, Thymol, α-Tocopherol, Tonka Undecanone, Undecanal, Valencene, Valeraldehyde/Pentanal, Verdoxan, α-Ylangene, Umbelliferone, Valencene or Vanillin.

In some cases, the composition comprises a cannabinoid-stabilizing blend including at least two exogenous terpenes, terpenoids, or at least one terpene and at least one terpenoid. For example, the cannabinoid-stabilizing blend can include a mixture of 3, 4, 5, 6 or more terpenes and/or terpenoids, such as a mixture comprising Eucalyptol, α-Terpinene, Limonene, and p-Cymene, or δ-Terpinene (AKA terpinolene), Geraniol, Carvone, Linalool, and Thymol. The cannabinoid-stabilizing blend can include a specific terpene and/or terpenoid in any concentration, typically about 0.01-25% by weight of the mixture. The blend may be composed of an equal or unequal portion of each exogenous terpene/terpenoid. The blend can further include a suitable carrier, such as MCT or other inert oil.

Another aspect of the present disclosure includes a method of determining the one or more terpenes, terpenoids, or blend thereof, that will stabilize the content of at least one of the cannabinoids of the product for a desired duration, after exogenous exposure thereto. Stability can be assessed by comparing the initial value and a value after a predetermined storage duration. The content can be considered stabilized when the initial value is substantially similar, substantially the same, or the same as a subsequent value. The stabilizing effect can also be determined relative to a control. For example, the THC content of a stabilized cannabinoid-containing product can be up to 50% greater than the THC content of a corresponding cannabinoid-containing product stored without the terpene, terpenoid, or blend thereof under the same conditions. Determination of cannabinoid content can be made by the skilled artisan using routine measurements (e.g., liquid chromatography, HPLC, tandem mass spectrometry). The duration can be based on the expected shelf-life of a cannabinoid-containing product under normal use and will vary based on the product type. The method can include subjecting the cannabinoid-containing product to real-time stability tests or accelerated storage tests to determine the change in cannabinoid content under typical storage conditions for the product with and without the selected terpene, terpenoid, or blend thereof at one or more concentrations. Testing of blends can include mixing terpenes, terpenoids, or both in various proportions according to standard food/laboratory practices. Real-time tests can be performed on products stored according to the recommended temperature conditions. For example, products can be tested at 0, 3, 6, 9, and 12 months for the first year. Accelerated storage conditions include enhanced conditions to cause an accelerated degradation of a product. For example, a product traditionally stored at 25° C. in an airtight, opaque container may have studies performed at an elevated temperature, elevated relative humidity, exposure to light, and/or in the presence of other degradation promoting agent (e.g., an oxidizing agent) for 2-6 months.

The cannabinoid-stabilizing effect of a terpene, terpenoid, or blend thereof can be based on its ability to maintain the concentration of at least one cannabinoid within a specified range over time (e.g., about 90% of the initial value). The cannabinoid-containing product can include one or more of Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC), Tetrahydrocannabinolic acid C4 (THCA-C4), Tetrahydrocannbinol C4 (THC-C4), Tetrahydrocannabivarinic acid (THCVA), Tetrahydrocannabivarin (THCV), Tetrahydrocannabiorcolic acid (THCA-C1), Tetrahydrocannabiorcol (THC-C1), Delta-7-cis-iso-tetrahydrocannabivarin, Δ8-tetrahydrocannabinolic acid (Δ8-THCA), Δ8-tetrahydrocannabinol (Δ8-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 10-Ethoxy-9-hydroxy-Δ6a-tetrahydrocannabinol, 8,9-Dihydroxy-Δ6a(10a)-tetrahydrocannabinol (8,9-Di-OH-CBT-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Isocanabinoids, Epigallocatechin gallate, Dodeca-2E, 4E,8Z,10Z-tetraenoic acid isobutylamide, or Dodeca-2E,4E-dienoic acid isobutylamide. In some cases, the concentration of tetrahydrocannabiniol in acid or decarboxylated form (THCA or THC, respectively), cannabidiol in acid or decarboxylated form (CBDA or CBD, respectively), cannabigerol in acid or decarboxylated form (CBGA or CBG, respectively), cannabichromene in acid or decarboxylated form (CBCA or CBC, respectively) tetrahydrocannabivarin in acid or decarboxylated form (THCVA or THCV, respectively), Cannabidivarin in acid or decarboxylated form (CBDVA or CBDV respectively) or cannabinol in acid or decarboxylated form (CBNA or CBN, respectively) is stabilized by the presence of the terpenoid. For example, the exogenous terpene, terpenoid, or blend thereof can be effective for stabilizing the total THC content in the cannabinoid-containing product such that less than 6% w/w of the initial THC content is lost during storage.

The present disclosure also includes methods of improving the stability of at least one cannabinoid of a cannabinoid-containing product during storage using one or more exogenous terpenes, terpenoids, or a blend thereof. For example, the method can include contacting the cannabinoid-containing product with an exogenous terpene, terpenoid, or blend thereof having a stabilizing effect, as discussed above. In some cases, contacting includes exposing the cannabinoid-containing product to the headspace of the exogenous terpene, terpenoid, or blend thereof (i.e., at least some of the terpene, terpenoid, or blend thereof is present in a vapor phase). Alternatively or additionally, contacting includes applying, mixing, infusing, or incorporating the terpene, terpenoid, or blend thereof onto or into the cannabinoid-containing product. In some cases, the contacting occurs during preparation, manufacture, or packaging of the product. The amount of terpene, terpenoid, or blend thereof in, on, or with the product can be based on the weight of the total product or the weight of the at least one cannabinoid. For example, the method can include adding at least 0.01% by weight of the exogenous terpene, terpenoid, or blend thereof to the product. In some cases, the method includes adding about 0.01-10%, 0.1-5%, 0.5-3.25% or about 0.01, 0.05, 0.10, 0.25, 0.50, 1.0, 1.5, 2, 2.5, 3, 3.5 or 4% by weight of the exogenous terpene, terpenoid, or blend thereof based on the total weight of the product.

### Cannabinoid-Stabilizing Packaging

In one or more embodiments described above, the cannabinoid-containing product and the one or more exogenous terpenes, terpenoids, or blend thereof, are packaged in a storage container. Thus, another aspect of the present disclosure includes a system for stabilizing at least one cannabinoid of a cannabinoid-containing product using an exogenous terpene, terpenoid, or blends thereof.

The system includes a suitable storage container. The storage container can be any size appropriate for the quantity of cannabinoid-containing product to be stored (e.g., a bulk storage container (1-100 kg), multi-dose container, single-use storage container). A suitable storage container can be a jar-style package for cannabinoid-containing product as described in US20210163194A1, the contents of which are incorporated herein by reference. The jar-style packaging generally has a main storage compartment for holding the cannabinoid-containing product, a cap/lid, and a dispersion matrix containing the one or more exogenous terpenes and/or terpenoids. In some cases, the cap/lid has an underside for holding or affixing the dispersion matrix thereto. The one or more exogenous terpenes and/or terpenoids can be infused into the dispersion matrix. The dispersion matrix can undergo secondary packaging, such as, in a packet or capsule for example. The cannabinoid-containing product can be packed with the dispersion matrix such that both are in communication with the air in the package. The dispersion matrix can be placed inside the package but not affixed to the package. This style of dispersion matrix insert may take any form known to those having skill in the art and particularly contemplates packets and capsules.

In some cases, a jar style package for holding cannabinoid-containing product having at least one cap/lid can include a primary container portion which is used to hold the cannabinoid-containing product and a secondary compartment which is used to hold the dispersion matrix. The primary and secondary compartments can be spatially arranged in any configuration, with a permeable barrier permitting vapor or volatiles in the dispersion matrix to permeate the air in the primary container. The secondary compartment can have a secondary cap to enable the placement and sealing off of the dispersion matrix.

The system can include a dispersion matrix insert that has an adhesive component. The adhesive component enables the dispersion matrix insert to be affixed to the inside of virtually any type of package used to hold the cannabinoid-containing product. In some embodiments, the adhesive style dispersion matrix insert is affixed to the package facing surface of a cap for the package.

In some cases the storage container is a bag style package incorporating a dispersion matrix insert. The dispersion matrix insert can be placed inside the bag style package. In an embodiment, the bag has a front side and a back side wherein the back side of the bag is perforated and the volatiles insert is placed over the perforations.

The dispersion matrix can be configured as set forth in US20210163194A1. For example, in some embodiments the dispersion matrix is made up of a core comprising the one or more terpenes and terpenoids, with the core disposed within a housing and/or barrier layer. In some such embodiments, the core may be manufactured within the housing so that a solution comprising the one or more exogenous terpenes and/or terpenoids can later be injected into the core. In some embodiments the core is made up of an absorbent material such as blotter paper or the like. In some embodiments the core is formulated as an oil based gel, such as a solid or semisolid gel. In some embodiments the dispersion matrix insert is a capsule, having a dispersion matrix core impregnated with solution comprising the one or more terpenes and terpenoids and disposed between a top half and a bottom half of the capsule shell. The dispersion matrix can be seated within the capsule body. In some embodiments the dispersion matrix insert is a packet or a laminate.

The dispersion matrix can include at least one material that acts as a reservoir for a terpene, terpenoid, or blends thereof, permitting controlled release over time. For example, the matrix can include a laminate, silicone rubber, polymers, and combinations thereof known in the art for use in controlled release matrices. In some embodiments the dispersion matrix insert contains is a silicone rubber into which the exogenous terpene, terpenoid, or blend thereof has been impregnated.

In some embodiments, the dispersion matrix includes a material capable of absorbing a stabilizing composition of terpene, terpenoid, or blends thereof in a carrier oil. For example, the absorbent material can be a woven or non-woven material such as cellulose, rayon, cotton, or other polymeric material adapted for readily absorbing oil based solutions. In some embodiments, the absorbent material is loaded with the stabilizing composition and then laminated between layers of a permeable barrier film that will transmit vapors emitted by the composition. The vapor transmission rate can be optimized by the type of barrier used, its thickness and porosity, as well as the area of the barrier relative to the absorbent material.

In some embodiments the dispersion matrix is a multi-laminate sustained release device capable of releasing vapor at a controlled rate for a prolonged period of time and having a first layer of a pressure sensitive adhesive release liner for providing a protective peel strip for the device, a second layer of silicone pressure sensitive adhesive for adhering the device to a substrate to which it is applied, a third layer of a volatiles impregnated matrix of a silicone material selected from the group consisting of silicone elastomers, silicone elastomers having adhesive characteristics, and elastomeric silicone pressure sensitive adhesives, and a fourth layer of a permeable facestock backing member on the surface of the device for controlling the rate of release of the volatiles from the impregnated matrix.

The dispersion matrix insert can include indicia disposed on an outer surface of the dispersion matrix insert covering. The indicia can include artwork, a logo, an advertisement, an instruction, a brand, a trademark, etc. The indicia can be formed by printing on an outer surface, molded into the material of the outer surface, etc.

In some embodiments, the system further includes a humidity controlling element. For example, the dispersion matrix can be coupled with a humidity controlling element such as that described in US patent publication US20150328584 A1, which is incorporated herein in its entirety. In such embodiments, dispersion matrix may be a dual layered design wherein the humidity controlling element may be stacked on top or below the dispersion matrix, or be disposed side-by-side with the dispersion matrix.

In one or more embodiments, the system includes a cap for securing the dispersion matrix. For example, the cap is removably secured to an open end of a jar or bottle to close the main storage compartment. The cap can include a dispersion matrix compartment underside (i.e., facing the main storage compartment). A permeable support defines a lower boundary of the dispersion matrix compartment. One or more dispersion matrix inserts, as described above, are retained within dispersion matrix compartment by the permeable support and the underside of the cap. For example, the dispersion matrix insert can be one or more sealed layers containing the terpene, terpenoid, or blend thereof, or a loose mixture of elements/compounds comprising the exogenous terpene, terpenoid, or blend thereof adapted to be retained within the dispersion matrix compartment. The permeable support can include a plurality of apertures to allow for communication between the dispersion matrix compartment and the main storage compartment. The permeable support can be held in place against the cap interior when the cap is secured to the jar/bottle.

In some cases, the permeable support is configured to hold the dispersion matrix insert within a downwardly extending well portion which extends into the main storage compartment when the cap and jar/bottle are secured together, as described in US20210163194A1.

### Examples

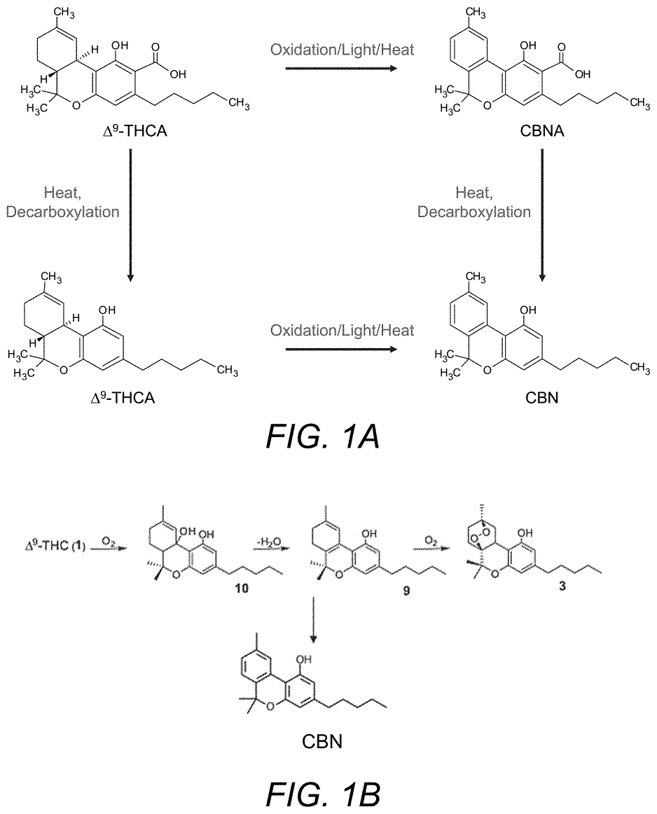

As shown in FIGS. 1A-B, THCA and THC are susceptible to oxidation, which can result in loss of total THC content in cannabis-containing products over time such that loss of total THC content over time is expected. In addition to enhancing the flavor and aroma profile of food, terpenes/terpenoids have been used to reduce bacterial contamination, inhibit lipid oxidation, extend shelf life and improve food quality. The following study was undertaken to assess whether exogenously applied terpenes could be used to improve cannabis product quality, cannabinoid concentration and potency during storage.

Blends of exogenous terpenes/terpenoids were first identified as having antioxidant activity (TABLE 1), and then assessed for a stabilizing effect that would reduce the loss of THC in dried cannabis flower during storage conditions. Compared with Blend 1, Blend 2 is relatively non-volatile, with each component having an approximate vapor pressure of less than 1 mm Hg at 25° C. (i.e., from 0.74 to 0.02).

TABLE 1
Terpene Blends

| Blend | Terpene Components | Type | Composition |
|---|---|---|---|
| 1 | Eucalyptol | monoterpenoid | 25 ± 0.2% (w/w) |
| | α-Terpinene | monoterpene | |
| | Limonene | monoterpene | |
| | p-Cymene | monoterpene | |
| 2 | δ-Terpinene | monoterpene | 20 ± 0.9% (w/w) |
| | (AKA terpinolene) | | |
| | Geraniol | monoterpenoid | |
| | Carvone | monoterpenoid | |
| | Linalool | monoterpenoid | |
| | Thymol | monoterpenoid | |

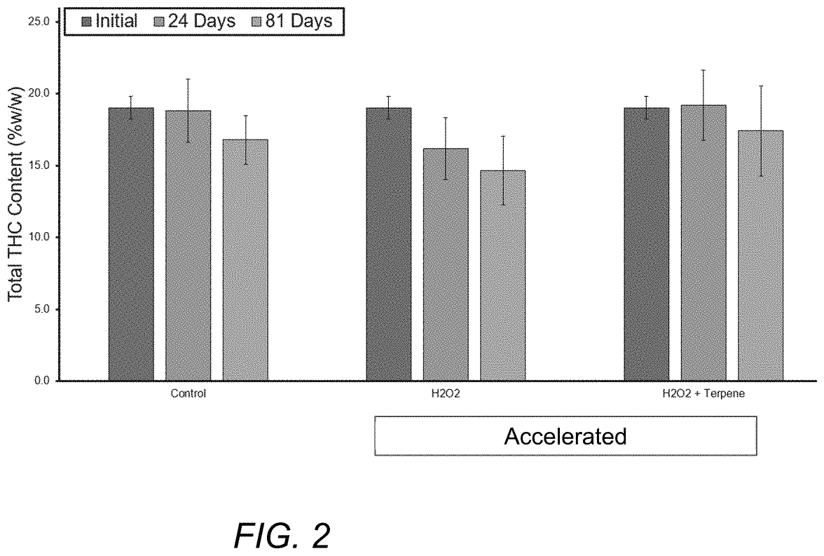

After analyzing initial cannabinoid content, an amount of terpene blend 1 was added to 3.5 (±0.05) g of ground flower (1, 2, or 3%, by weight of the dried cannabis) was applied to the product and stored in an air-tight container protected from light. All experiments were performed in triplicate. The testing conditions included: (1) Non-Accelerated Storage Conditions without exogenous terpenes “Control”; (2) Accelerated Conditions (+3.0% by weight hydrogen peroxide, initially, and refreshed each week) without exogenous terpenes, and (3) Accelerated Conditions with exogenous terpenes “H₂O₂+Terpene” (Blend 1). (FIG. 2). Butylated hydroxytoluene (BHT), a preservative used in food, known to prevent radical-mediated oxidation was employed as a positive control. Each container was opened twice/week and stirred, simulating usual conditions for access of medicinal cannabis. The cannabinoid content was analyzed after 24 and 81 days of storage.

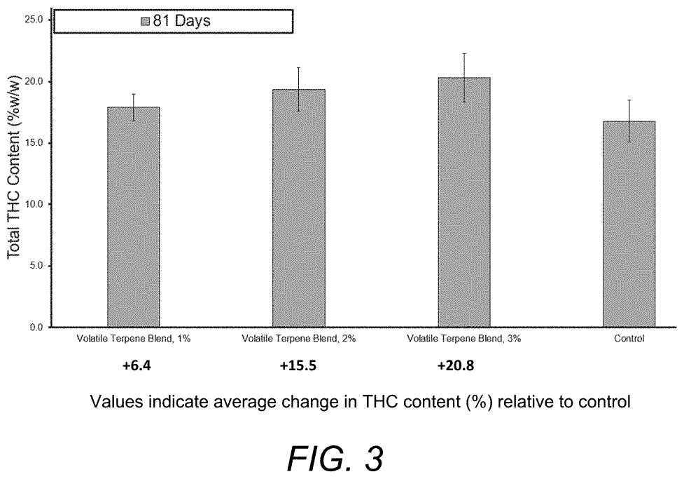

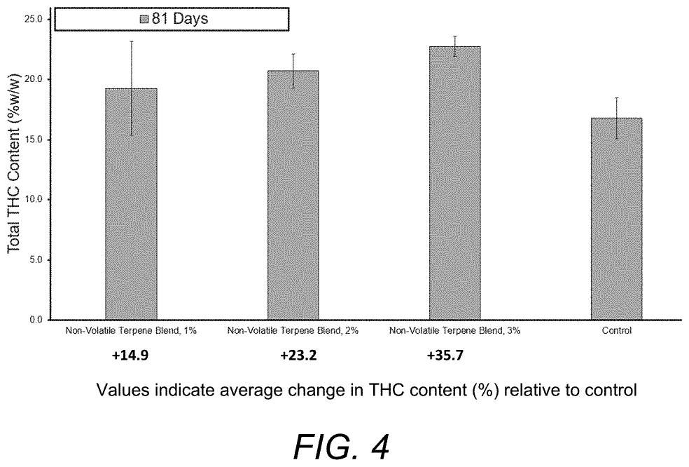

As shown in FIG. 2, application of the terpene blend slowed or prevented loss of total THC under non-accelerated and accelerated conditions. Unexpectedly, the terpene blend was more effective for stabilizing THC content than BHT. FIGS. 3 and 4 show a concentration-dependent relative increase in THC content (%) over control with volatile Terpene Blend (Blend 1) and non-volatile Terpene Blend (Blend 2), respectively. The stabilizing effect was observed after 81 days (TABLE 2) and also between days 24 and 81 (TABLE 3).

TABLE 2

| | Relative % Loss |
|---|---|
| SAMPLE | 0 to 81 Days |
| H₂O₂ | 23.06 |
| CONTROL | 11.76 |
| H₂O₂ + Terpene | 8.52 |
| BHT | 7.90 |
| Volatile Terpene Blend (Blend 1), 1% | 5.92 |
| Non-Volatile Terpene Blend (Blend 2), 1% | −1.24 |
| Volatile Terpene Blend (Blend 1), 2% | −1.75 |
| Volatile Terpene Blend (Blend 1), 3% | −6.80 |
| Non-Volatile Terpene Blend (Blend 2), 2% | −8.84 |
| Non-Volatile Terpene Blend (Blend 2), 3% | −19.57 |

TABLE 3

| | Relative % Loss |
|---|---|
| SAMPLE | 24 to 81 Days |
| BHT | 18.65 |
| CONTROL | 10.73 |
| H₂O₂ | 9.49 |
| H₂O₂ + Terpene | 9.40 |
| Volatile Terpene Blend (Blend 1), 1% | 9.12 |
| Non-Volatile Terpene Blend (Blend 2), 3% | 6.80 |
| Volatile Terpene Blend (Blend 1), 2% | 3.09 |
| Non-Volatile Terpene Blend (Blend 2), 1% | 0.27 |
| Non-Volatile Terpene Blend (Blend 2), 2% | −2.29 |
| Volatile Terpene Blend (Blend 1), 3% | −5.10 |

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. A method of improving stability of at least one cannabinoid of a cannabinoid-containing product during storage comprising:
(a) identifying a cannabinoid-containing product that will be stored for a predetermined duration;
(b) contacting the cannabinoid-containing product with one or more exogenous terpenes, terpenoids, or a blend thereof; and
(c) storing the cannabinoid-containing product for the predetermined duration;
whereby a final content of at least one cannabinoid after storing is substantially similar to an initial content of the at least one cannabinoid.

2. The method of claim 1, wherein the exogenous terpene or terpenoid is selected from the group consisting of 7,8-dihydroionone, Acetanisole, Acetyl Cedrene, Anethole, Anisole, Benzaldehyde, Bergamotene (α-cis-Bergamotene) (α-trans-Bergamotene), α-Bisabolol, Bornyl acetate, Borneol, Butanoic, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), D-Limonene, Decanal, Dehydrovomifoliol, Diallyl Disulfide, Dihydroactinidiolide, Dimethyl Disulfide, Eicosane/Icosane, Elemene (β-Elemene), Estragole, Ethyl acetate, Ethyl Cinnamate, Ethyl maltol, Eucalyptol, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10), 11-diene, Guaiol, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (β-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, α-Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpineol, α-Terpinene, γ-Terpinene, Terpinolene, Thiophenol, Thujone, Thymol, α-Tocopherol, Tonka Undecanone, Undecanal, Valencene, Valeraldehyde/Pentanal, Verdoxan, α-Ylangene, Umbelliferone, Valencene and Vanillin, or a blend thereof comprising at least two terpenes, at least two terpenoids, or at least one terpene and at least one terpenoid.

3. The method of claim 1, wherein the cannabinoid-containing product is contacted with a blend including a mixture of at least four exogenous terpenes, terpenoids, or a combination thereof.

4. The method of claim 1, comprising adding 0.01-5% one or more exogenous terpenes, terpenoids, or a blend thereof by weight of the cannabinoid-containing product.

5. The method of claim 1, wherein the cannabinoid-containing product is contacted with a mixture of volatile terpenes and terpenoids, optionally comprising Eucalyptol, α-Terpinene, Limonene, and p-Cymene, or a mixture of non-volatile terpenoids, optionally comprising δ-Terpinene, Geraniol, Carvone, Linalool, and Thymol.

6. The method of claim 1, wherein the duration of storage is at least 2 months.

7. The method of claim 1, wherein the at least one cannabinoid is selected from the group consisting of tetrahydrocannabiniol in acid or decarboxylated form, cannabidiol in acid or decarboxylated form, cannabigerol in acid or decarboxylated form, cannabichromene in acid or decarboxylated form, tetrahydrocannabivarin in acid or decarboxylated form, Cannabidivarin in acid or decarboxylated form, and cannabinol in acid or decarboxylated form.

8. The method of claim 1, wherein contacting includes exposing the cannabinoid-containing product to a headspace comprising the terpene, terpenoid, or blend thereof for the duration of storage.

9. The method of claim 1, wherein contacting occurs during preparation, manufacture, or packaging of the cannabinoid-containing product.

10. A dispersion matrix for use in packaging comprising an exogenous terpene, terpenoid, or a blend thereof configured to improve stability of at least one cannabinoid of a cannabinoid-containing product during storage.

11. The dispersion matrix of claim 10, further comprising an adhesive component configured to affix the matrix to an interior surface of a package adapted to hold the cannabinoid-containing product.

12. The dispersion matrix of claim 10, comprising a core containing the exogenous terpene, terpenoid, or blend thereof.

13. The dispersion matrix of claim 12, wherein the core is formulated as an oil based gel or impregnated with solution comprising the exogenous terpene, terpenoid, or blend thereof.

14. The dispersion matrix of claim 12, wherein the core is seated within a capsule body.

15. The dispersion matrix of claim 11, further comprising at least one material configured to controllably release the exogenous terpene, terpenoid, or blend thereof, over time.

16. The dispersion matrix of claim 15, wherein the at least one material is a laminate, silicone rubber, or a control release polymer.

17. The dispersion matrix of claim 16, wherein the at least one material is a multi-laminate sustained release device capable of releasing vapor at a controlled rate for a prolonged period of time.

18. A system for storing a cannabinoid-containing product, the system comprising:
(a) a storage container configured to hold a cannabinoid-containing product;
(b) a dispersion matrix comprising an exogenous terpene, terpenoid, or blend thereof for improving stability of at least one cannabinoid; and
(c) a lid to seal the storage container;
wherein the dispersion matrix and the cannabinoid-containing product are in communication with air in the storage container allowing the exogenous terpene, terpenoid, or blend thereof to be in contact with the cannabinoid-containing product.

19. The system of claim 18, further including a humidity controlling element.

20. The system of claim 18, wherein the lid is configured to secure the dispersion matrix in the storage container.
